(19) United States
(12) Patent Application Publication
(10) Pub. No.: US 2022/0202996 A1
(43) Pub. Date: Jun. 30, 2022

(54) MEDICAL IMPLANT SURFACE-MODIFIED WITH FUNCTIONAL POLYPEPTIDE

(71) Applicants: SEOUL NATIONAL UNIVERSITY HOSPITAL, Seoul (KR); SOOKMYUNG WOMEN'S UNIVERSITY INDUSTRY-ACADEMIC COOPERATION FOUNDATION, Seoul (KR)

(72) Inventors: Chan Yeong HEO, Seongnam-si (KR); Sun Young NAM, Seongnam-si (KR); Mi Ji LEE, Seongnam-si (KR); Byung Ho SHIN, Seongnam-si (KR); Dong Sik SHIN, Seoul (KR); Seong Soo KIM, Seoul (KR)

(73) Assignees: SEOUL NATIONAL UNIVERSITY HOSPITAL, Seoul (KR); SOOKMYUNG WOMEN'S UNIVERSITY INDUSTRY-ACADEMIC COOPERATION FOUNDATION, Seoul (KR)

(21) Appl. No.: 17/609,585

(22) PCT Filed: Dec. 11, 2019
(86) PCT No.: PCT/KR2019/017471

§ 371 Date: Nov. 8, 2021

(30) Foreign Application Priority Data
May 8, 2019 (KR) 10-2019-0053898

(51) Int. Cl. A61L 27/54; A61K 38/20; A61L 27/26

(52) U.S. Cl. CPC A61L 27/54; A61K 38/2086; A61K 38/2026; A61L 2430/04; A61L 2300/802; A61L 2300/252; A61L 2300/41; A61L 27/26

(57) ABSTRACT

Provided is a medical implant including: an implant base having a surface made of a silicon material; a linker having one end attached onto the surface of the implant base; and a cytokine bound to another end of the linker. By inducing the secretion of anti-inflammatory cytokines, a capsular contracture, which is one of the complications that may occur after transplantation of the patient's breast implant, may occur less.

### TECHNICAL FIELD

One or more embodiments relate to a medical implant capable of inducing an anti-inflammatory response due to the surface-modification with a functional polypeptide. This application claims priority to Korea Patent Application No. 10-2019-0053898, filed on May 8, 2019, the disclosure of which is incorporated by reference herein in its entirety.

### BACKGROUND ART

The most common local complication associated with implants made of silicon material is capsular contracture. Capsular contracture accounts for 10.6% of complications, which may occur especially in patients who have undergone breast augmentation. Capsular contracture may cause a fibrous foreign body reaction and pain, due to various factors that promote hardening and tightening of a film at the contact site between the tissue and the implant.

Although the pathogenesis of capsular contracture has not been fully identified, the cellular composition of the implant film, including macrophages, fibroblasts, and lymphocytes, appears to promote the progression of fibrous globular formation. Therefore, it is necessary to develop an implant that does not cause capsular contracture and reduces the possibility of inflammation by inducing anti-inflammatory responses.

### DESCRIPTION OF EMBODIMENTS

### Technical Problem

One or more embodiments include a medical implant including: an implant base having a surface made of a silicon material; a linker having one end attached onto the surface of the implant base; and a cytokine bound to another end of the linker.

Other objectives and advantages of the present application will become more apparent from the following detailed description in conjunction with the appended claims and drawings. Content not described in this specification will be omitted because it can be sufficiently recognized and inferred by those skilled in the technical field or similar technical field of the present application.

### Solution to Problem

Descriptions and embodiments provided in this application may also be applied to other descriptions and embodiments. That is, all combinations of the various elements disclosed in this application fall within the scope of this application. In addition, it shall not be seen that the scope of the present application is limited by the detailed description described below.

One or more embodiments include a medical implant including: an implant base having a surface made of a silicon material; a linker having one end attached onto the surface of the implant base; and a functional polypeptide bound to another end of the linker.

The term “implant” used herein refers to all transplantable materials or implants that can be used for skin depressions or dents due to wrinkles, etc., and can be used to improve volume for cosmetic purposes. The term “medical implant” includes implants that restore human tissue when the human tissue is lost, and medical devices or instruments that are temporarily or permanently introduced into mammals to prevent or treat abnormal medical reactions. In addition, the implants may include any that is introduced subcutaneously, transdermally, or surgically and is left in organs such as arteries, the veins, ventricles, and the atria, or the tissues or lumens of organs.

The basic material of the implant may include one selected from ultra-high molecular weight polyethylene (UHMWPE), poly ether ether ketone (PEEK), polyurethane, silicon elastomer, bioabsorbable polymer, aluminum oxide, zirconium, physiologically active glass fiber, silicon nitrogen compounds, calcium phosphate, and carbon.

Silicone may be a polymer based on a bond between silicon and oxygen (—Si—O—Si—O—). A siloxane bond (—Si—O—Si—O—) is formed when methyl chloride (CH₃Cl) is reacted with crystalline silicon to synthesize dimethyldichlorosilane and then hydrolyzed. According to this polymerization method, various kinds of polymers can be synthesized. A typical example is a silicone resin composed of linear polydimethylsiloxane and oligosiloxane molecules. Silicon is a colorless and odorless insulator that oxidizes slowly and is stable at high temperatures. Silicon may be used in lubricants, adhesives, gaskets, and molding artificial prostheses. The term “linker” used herein refers to a linkage that connects two different fusion partners (for example, biological polymers, etc.) using a covalent bond. The linker may be a peptide linker or a non-peptide linker, and in the case of a peptide linker, the linker may consist of one or more amino acids.

The term “functional polypeptide” used herein refers to a polypeptide having a biological function or activity that is identified through a definitive functional assay and is associated with a specific biological, morphological, or phenotypic change in a cell. The functional polypeptide may be derived from any species. The functional polypeptide may be either in the native or non-natural form thereof. A native functional polypeptide refers to a peptide that exists in nature. On the other hand, a non-natural functional polypeptide refers to a mutant polypeptide derived by introducing an appropriate mutation (addition, deletion, or substitution of amino acids) to an amino acid sequence as long as the unique function of the functional peptide is maintained based on the amino acid sequence of the native functional polypeptide. In an embodiment, the functional polypeptide may be one or more selected from proteins, cytokines and chemokines.

According to an embodiment, the medical implant may suppress the inflammatory response that may occur in a subject upon contact with the implant because a functional polypeptide, such as IL-4, attached onto the surface of the implant induces the differentiation of anti-inflammatory cytokines. Therefore, the medical implant may replace the existing implant that may cause inflammation.

The term “cytokine” used herein is a small cell-signaling protein molecule secreted by a plurality of cells, and refers to a signaling molecule widely used for information exchange within a cell. Cytokine may include monokines, lymphokines, traditional polypeptide hormones, etc., and may include tumor necrosis factor-α (TNF-α), tumor necrosis factor-β (TNF-β), transforming growth factor (TGF) (for example, TGF-α or TGF-β), interferon-α (IFN-α), interferon-β (IFN-β), interferon-γ (IFN-γ), Interleukin-1 (IL-1), IL-1α, IL-2, IL-3, IL-4, IL-5, IL-6, IL-7, IL-8, IL-9, IL-10, IL-11, IL-12, or IL-13. Cytokines may also include recombinant cell cultures and biologically active equivalents of a cytokine from natural sources or of native sequence cytokines.

The term “chemokine” used herein refers to a basic heparin-binding small-molecule protein that acts on leukocytic emigration and activation. There are four cysteine residues in the chemokine molecule, and the chemokine can be classified into four subfamilies: CXC(CXCL), CC(CCL), CX3C(CX3CL) and C(XCL) according to the type of existence of the first two cysteine residues in the molecule. Currently, more than 40 species have been identified.

In an embodiment, the surface of the implant base may include a shell made of a silicon material.

The term “shell” refers to an external part of an implant made to enable organ transplantation, and refers to a pouch in which a fluid material can be filled in the shell.

In an embodiment, the cytokine may include at least one selected from IL-4, IL-10, and IL-13.

IL-4 is a cytokine that induces differentiation of inactive helper T cells (Th0 cells) into Th2 cells. Th2 cells activated by IL-4 may produce additional IL-4. Th2 cells may secrete IL-4 to develop M2 macrophages. The “macrophages” used herein are cells that are distributed in all tissues in a living body and responsible for immunity, and refer to cells involved in the removal of invading pathogens, removal of virus-infected autologous cells and cancer cells, and induction of an inflammatory response. Macrophages can be categorized according to the process of development: tissue-resident macrophages differentiated from the yolk sac or fetal liver, and monocyte-derived macrophages differentiated from monocytes (differentiated from bone marrow cells) in the blood by inflammatory reactions or pathogen invasion. Tissue-resident macrophages and monocyte-derived macrophages may be differentiated, by cytokines, into M1 macrophages and M2 macrophages that act on different immune responses. M1 macrophages are induced by IFN-γ and TNF-α, which are cytokines of Th1 cells, and act on induction of Th1 response, induction of inflammatory response, inhibition of cancer growth, and the like. M2 macrophages are induced by IL-4, IL-10, etc., which are cytokines of Th2 cells, and may act in Th2 response induction, inflammatory response inhibition, damaged tissue repair, and the like. The increase in M2 macrophages is associated with the secretion of IL-10 and TGF-β, and as a result, pathological inflammation may be reduced.

IL-10 is a functional Th2 cell cytokine, which has the functions of replication of M1 macrophages, monocytes, and T-cell lymphocyte, and inhibition of secretion of inflammatory cytokines (IL-1, TNF-α, TGF-β, IL-6, IL-8, and IL-12). In an embodiment, the linker may be represented by Formula 1.

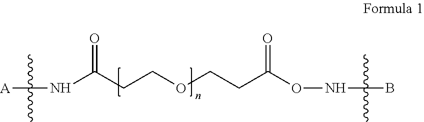

wherein, in Formula 1,

A is an implant with a silicon surface,

B is the end of the functional polypeptide, and

n is an integer from 5 to 15.

In an embodiment, the medical implant may induce the secretion of anti-inflammatory cytokines. Cytokines such as IL-4 or IL-10 may be attached onto the surface of the medical implant, which is associated with the activity of M2 macrophages as described above. When the M2 macrophages are increased, the secretion of anti-inflammatory cytokines such as IL-10 and TGF-β may be induced, so that the medical implant may suppress the inflammatory response.

In an embodiment, the process of attaching the functional peptide onto the surface of the medical implant having a silicon material may be performed by the process illustrated in FIG. 1. For example, the process may include forming a hydroxyl group (—OH) on the surface of the silicon material by treating the medical implant having a silicon material with oxygen plasma (O₂ plasma); sequentially adding APTMS and Bis-dPEG®₅-NHS ester onto the surface of the silicon material; and adding, under a weak alkaline condition, a functional polypeptide including a carboxyl group at the terminal thereof, for example, IL-4, to the surface of the silicon material to which the materials are added.

In this regard, in the process of treating the oxygen plasma, the amount of energy and the treatment time may be appropriately changed as long as a sufficient amount of hydroxyl groups can be introduced onto the surface of the silicon material. For example, the amount of energy may be about 1000 W to about 50 W, about 900 W to about 150 W, about 800 W to about 250 W, about 700 W to about 350 W, or about 600 W to 450 W, and the like, and the treatment time may be from about 30 minutes to about 2 minutes, about 25 minutes to about 4 minutes, about 20 minutes to about 6 minutes, about 15 minutes to about 8 minutes, about 10 minutes to about 9 minutes, etc., but is not limited thereto. In addition, in the process of sequentially adding APTMS and Bis-dPEG®5-NHS ester to the surface of the silicon material, the reaction time and the concentration may also be suitably changed depending on the purpose and aspect of use.

In an embodiment, the medical implant may be a breast implant.

The ‘breast implant’ may be an implant that can be implanted into a patient in breast-related surgery and treatment. The inside of the shell of the breast implant may be filled with saline, hydrogel, or silicone gel as the filler. In addition, the breast implant may be a round-type implant and an anatomical-type implant depending on the shape thereof, and may be a smooth-type implant and a texture-type implant depending on the surface condition.

In an embodiment, the breast implant may inhibit the formation of breast capsular contracture. The term “capsular contracture” used herein refers to a condition in which the film around the transplanted implant becomes thick and hard due to excessive fibrosis, and is one of the side effects occurring during transplantation. Macrophages may be the main cause of this capsular contracture at the site of the implant. IL-4 or IL-10 attached onto the breast implant may induce the activation of M2 macrophages and the secretion of anti-inflammatory cytokines thereby. In this way, the breast implant may inhibit the formation of breast capsular contracture.

In an embodiment, the breast implant may have an Rq value of about 4 nm to about 10 nm. For example, the Rq value may be from about 4 nm to about 9 nm, about 4 nm to about 8 nm, about 4 nm to about 7 nm, about 4 nm to about 6 nm, about 4 nm to about 5 nm, about 5 nm to about 9 nm, about 5 nm to about 8 nm, about 5 nm to about 7 nm, about 5 nm to about 6 nm, about 6 nm to about 9 nm, about 6 nm to about 8 nm, or about 6 nm to about 7 nm. The Rq value refers to the mean square roughness value. After the surface of the implant is treated with oxygen plasma, 3-aminopropyltrimethoxysilane (APTMS) and cytokines are added and attached thereonto to give effective roughness to the surface of the breast implant. Due to this surface roughness, the movement of the implant after transplantation may be prevented through attachment to the breast tissue, and the formation of capsular contracture may be suppressed.

### Advantageous Effects of Disclosure

A medical implant according to an aspect can inhibit the formation of capsular contracture, which is one of the complications that may occur in patients undergoing breast implant transplantation, by inducing the differentiation of anti-inflammatory cytokines by a functional polypeptide, such as IL-4, attached onto the surface of the implant.

### BRIEF DESCRIPTION OF DRAWINGS

FIG. 1 shows a diagram showing the process of attaching the cytokine to the surface of the implant.

FIG. 2 shows an image of the medical implant manufactured through the process illustrated in FIG. 1.

FIG. 3 is a graph of the nitrogen concentration of the silicon surface according to the treatment time when APTMS is bonded to the silicon surface.

FIG. 4A is a graph showing the roughness value (Rq) measured in untreated silicon and the surface thereof in three dimensions.

FIG. 4B is a graph showing the roughness value (Rq) measured after treating silicon with O₂ plasma and the surface thereof in three dimensions.

FIG. 4C is a graph showing Rq measured after treating silicon with O₂ plasma and APTMS and the surface thereof in three dimensions.

FIG. 4D is a graph showing Rq measured after treating silicon with O₂ plasma, APTMS, and Bis diPEG®₅ and the surface thereof in three dimensions.

FIG. 4E is a graph showing Rq measured after treating silicon with O₂ plasma, APTMS, Bis diPEG®₅, and IL-4 and the surface thereof in three dimensions.

FIG. 4F is a graph showing Rq measured after treating silicon with O₂ plasma, APTMS, Bis diPEG®₅, and IL-13 and the surface thereof in three dimensions.

FIG. 5 is a graph showing the viability (toxicity) of cells measured in a group having a smooth surface and a group having a smooth surface with IL-4 attached thereonto (smooth+IL-4 group).

FIG. 6A shows an image showing the results of Western blot performed to measure the degree of differentiation of M2 macrophages.

FIG. 6B shows an image showing the results of Western blot performed to measure the degree of differentiation of M1 macrophages.

FIG. 6C shows an image showing the results of immunofluorescence performed to measure the degree of differentiation of M2 macrophages.

FIG. 7 is a graph showing the results of an enzyme-linked immunosorbent assay (ELISA) performed to measure proinflammatory cytokines.

FIG. 8 is a graph showing the results of ELISA performed to measure anti-inflammatory cytokines.

FIG. 9 is an image showing the result of Western blot performed to determine whether the IL-4-modified implant alone can activate the STAT6 pathway.

FIGS. 10A and 10B are graphs and microscopic images showing the changed capsular thickness and collagen density after transplantation of the implant into a mouse as an animal experimental model for a medical implant according to an aspect.

FIG. 11A shows comparison results of the production amount of TNF-α through ELISA in a group having a smooth surface and a group having a smooth surface with IL-10 attached thereonto (smooth+IL-10 group).

FIG. 11B shows comparison results of the production amount of IL-6 through ELISA in the smooth group and the smooth+IL-10 group.

FIG. 11C shows comparison results of the production amount of IL-1β through ELISA in the smooth group and the smooth+IL-10 group.

FIG. 11D shows comparison results of the production amount of IL-10 through ELISA in the smooth group and the smooth+IL-10 group.

FIG. 11E shows comparison results of the production amount of IL-4 through ELISA in the smooth group and the smooth+IL-10 group.

FIG. 12 is a graph showing the viability of cells measured in the smooth group and smooth+IL-10 group.

FIG. 13 is an image showing the results of immunofluorescence measurement performed to measure the degree of differentiation of M2 macrophages in the smooth group and the smooth+IL-10 group.

FIG. 14 shows comparison results of Arg-1, IL-10, IL-1β, and TNF-α in a group having a smooth surface and a group having a smooth surface with IL-13 attached thereonto (smooth+IL-13 group).

FIG. 15 is a graph showing the viability of cells measured in the smooth group and the smooth+IL-13 group.

### MODE OF DISCLOSURE

Hereinafter, the present disclosure will be described in more detail through examples. However, these examples are for illustrative purposes of the present disclosure, and the scope of the present disclosure is not limited to these examples.

### REFERENCE EXAMPLE

### Reference Example 1. Cell Culture

RAW 264.7 cells were purchased from the American Type Culture Collection (Rockville, Md., USA) and incubated in a medium-supplemented with 10% heat-inactivated fetal bovine serum (FBS), 100 U/ml of penicillin, and 100 μg/ml of streptomycin (Gibco, Carlsbad, Calif.) under a humidified condition containing 5% CO₂ at a temperature of 37° C.

### Reference Example 2. Western Blot Analysis

After 24 hours, RAW 264.7 cells were dissociated with cold PBS and homogenized on ice using cell lysis buffer (Cell Signaling Technology, Danvers, Mass., USA). After heating the sample at a temperature of 95° C. for 5 minutes and cooling briefly on ice, 30 μg of protein was loaded onto a 10% SDS-PAGE polyacrylamide gel. After gel electrophoresis, the gel was transferred to a nitrocellulose membrane (GE Healthcare, Piscataway, N.J., USA). The membrane was blocked with 5% BSA in PBS for 2 hours at room temperature, and primary antibodies against iNOS (Abcam, Cambridge, UK), Arg-I (Santa Cruz, Calif., USA) and control GAPDH were incubated overnight at a temperature of 4° C. After washing 4 times with PBS-T (pH 7.4), the cell membrane was diluted 1:2,000 with HRP-conjugated an anti-mouse or anti-rabbit IgG secondary antibody (Santa Cruz Biotechnology, Santa Cruz, Calif., USA) and stored at room temperature for 2 hours. Next, the membrane was washed 4 times with PBS-T. A Western blot detection kit (EZ-Western Lumi pico, Dogen, Korea) was used for protein detection. Finally, protein was quantified in the blot, and analysis for densitometry of the blot was performed in Image J (Image J, National Institutes of Health, USA). Relative quantitation was calculated after being converted to GAPDH levels. The above analysis method was repeated twice.

### Reference Example 3. Immunofluorescence Assay

Cells were washed 3 times with PBS (pH 7.4) for 5 min each. Then, the slides were treated with a blocking solution (0.2% Triton X-100, 1% BSA in PBS) for 1 hour to block non-specific antigen bindings. The slides were then incubated overnight with diluted primary antibody. The next day, after washing 3 times with PBS, the plate was incubated at room temperature for 1 hour with a secondary antibody diluted 1:2000. Then, the slides were thoroughly washed with PBS and then staining was performed thereon using DAPI (DAPI, VECTASHIELD, Vector Laboratories, USA) to stain cell nuclei. Images were then taken using a z-stack with a confocal microscope.

### Reference Example 4. Reverse Transcription Polymerase Chain Reaction (RT-PCR) and Quantitative Real-Time Polymerase Chain Reaction (qRT-PCR)

RNA of RAW 264.7 cells was extracted according to the instructions of the RNA extraction kit (easy-BLUE RNA extraction kit, iNtRON Biotechnology, Gyeonggi-do, Korea). RNA was quantified with a spectrometer (Nanodrop 1000, Wilmington, Del.). From 2 μg of RNA, 20 μl of cDNA was synthesized using reverse transcriptase (AccuPower® RT PreMix, Bioneeer Corporation, Daejeon, Korea) according to the manufacturer's instructions. The reaction was performed using an ABI 7500 Real-Time PCR System (Applied Biosystems). The expression level of the gene was normalized using GAPDH mRNA. Expression levels presented were the mean values of each sample. In the case of RT-PCR, the annealing temperature for IL-6 and GAPDH was 62° C. The resultant product was electrophoresed on a 2% agarose gel and stained with ethidium bromide.

### Reference Example 5. Statistical Analysis Method

Each data was presented as mean±standard error (SEM). One-way ANOVA was used for multi-group comparisons after Tukey's test. Power analysis was applied to determine the difference between the control group and the treatment group. P<0.05 was considered as being significant.

### EXAMPLE

### Example 1. Preparation of IL-4 Surface-Modified Silicon

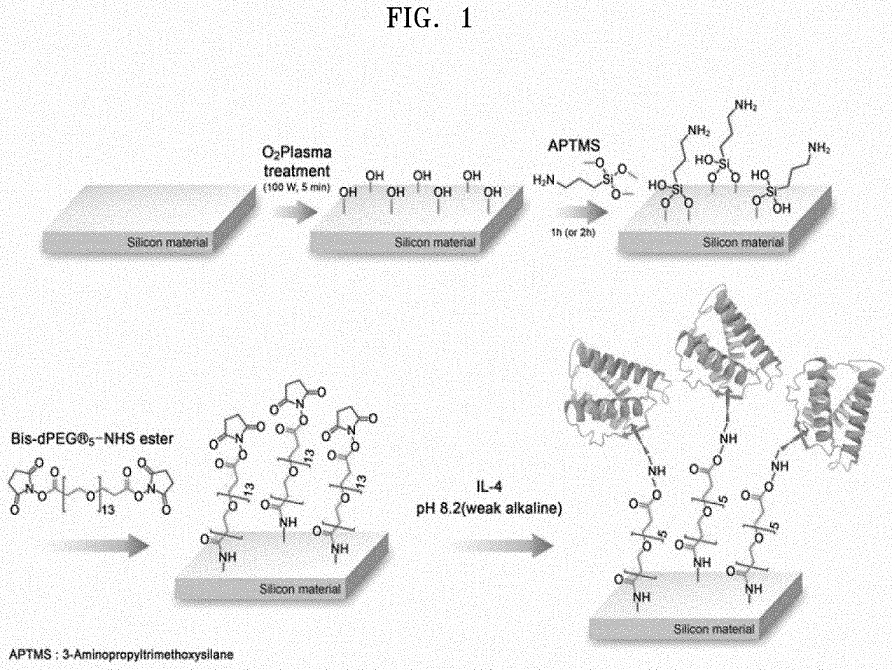

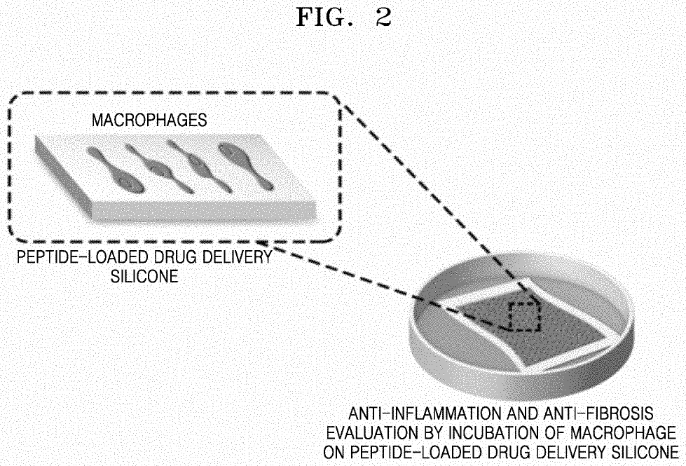

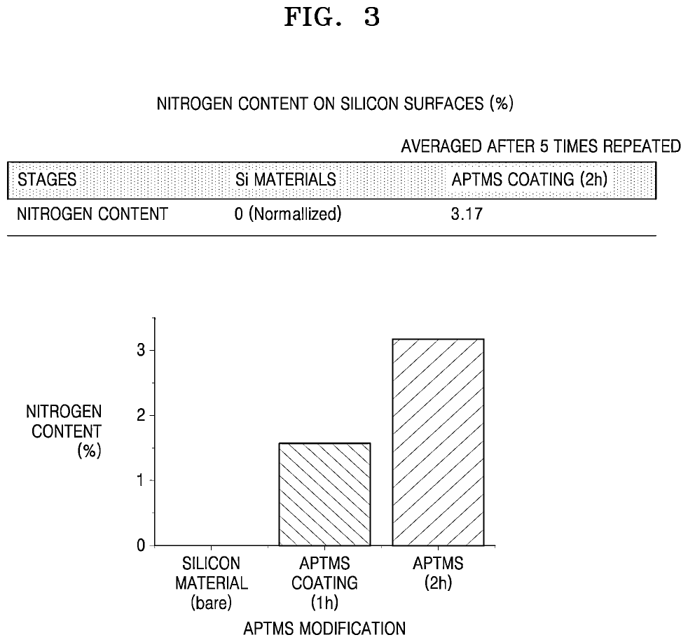

This embodiment was performed to manufacture a medical implant according to an aspect. FIGS. 1 and 2 schematically show the surface modification of the silicon and the production process of the cytokine, which is one of the functional polypeptides, attached thereonto. Specifically, the surface of the silicon was surface-treated with oxygen plasma (O₂ plasma) at 100 W for 5 minutes, and then, 3-aminopropyltrimethoxysilane (APTMS) was attached thereonto. APTMS attachment was performed for 1 or 2 hours. The concentration of nitrogen according to the coating time of APTMS is shown in FIG. 3.

As shown in FIG. 3, the concentration of nitrogen was increased as the coating time of APTMS was increased, and this result indicates that the modification of the silicon surface through APTMS proceeded normally.

After coating APTMS, bis-dPEG®5 NHS ester was added to modify the surface. As shown in FIG. 2, IL-4, one of the functional polypeptides, was finally introduced onto the modified surface in a weakly basic state of pH 8.2.

### Example 2. Contact Angle (WCA) Analysis

In this example, the contact angle was measured to measure the degree of modification of the silicon surface prepared by the method of Example 1. Specifically, as the contact angle, a water contact angle (WCA) was measured using a program (First Ten Angstroms FTA 1000 C Class) in which Sessile drop technology was combined with drop shape analysis software. To measure static advancing contact angles, 2.0 μL of water droplet was added to the droplet every 2 seconds to grow the droplet, and then the droplet was added and within 5 seconds, images thereof were captured. This procedure was repeated 20 times. For the concrete reliability of WCA, the contact angle of a non-ideal surface such as APTMS SAM was calculated using the tangent-leaning method. The WCA of each of: a sample including Si/O₂ plasma/APTMS/bis-dPEG®₅ NHS ester/IL-4, which was a surface-modified silicon prepared by the method of Preparation Example 1; a control sample including Si, Si/O₂ plasma, Si/02 plasma/APTMS, Si/O₂ plasma/APTMS/bis-dPEG®5 NHS ester, or Si/02 plasma/APTMS/bis-dPEG®₅ NHS ester/(IL-4 or IL-13), was measured. The WCA value is the average of at least three measurements. The measurement results are shown in Table 1.

TABLE 1

| Sample | WCA (°) |
|---|---|
| (a) Bare silicone prosthetic material (Si) | 93.90 |
| (b) Si/O₂ plasma | 0.16 |
| (c) Si/O₂ plasma/APTMS | 97.80 |
| (d) Si/O₂ plasma/APTMS/Bis diPEG_{@5} NHS ester | 100.60 |
| (e) (d)/IL4 (Cytokine immobilization) | 78.1 |
| (f) (d)/IL13 (Cytokine immobilization) | 76.6 |

As shown in Table 1, the untreated silicon surface had strong hydrophobicity, and thus, a large WCA value was measured therefor. When the silicon surface was formed with oxygen plasma, due to the enhanced hydrophilicity caused by the presence of OH— functional groups, the WCA value was decreased rapidly to 0.16°. In addition, as APTMS was introduced to the surface, the WCA value was 97.80°, that is, the hydrophobicity became stronger. After introduction of bis-diPEG®5 NHS ester, the WCA value was 100.60°, that is, the hydrophobicity was further enhanced. Additionally, the WCA values corresponding to the case in which functional polypeptides, IL-4 or IL-13 were introduced, were 78.1° C. and 76.6° C., respectively. That is, hydrophobicity was slightly attenuated, which is considered to be due to the hydrophilicity of cytokines. These WCA values and the changes thereof indicate that the silicon surface was modified step by step.

### Example 3. AFM Analysis

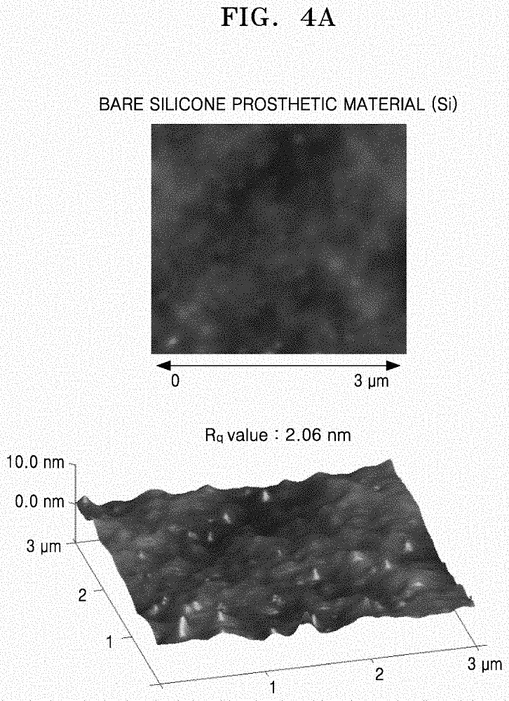

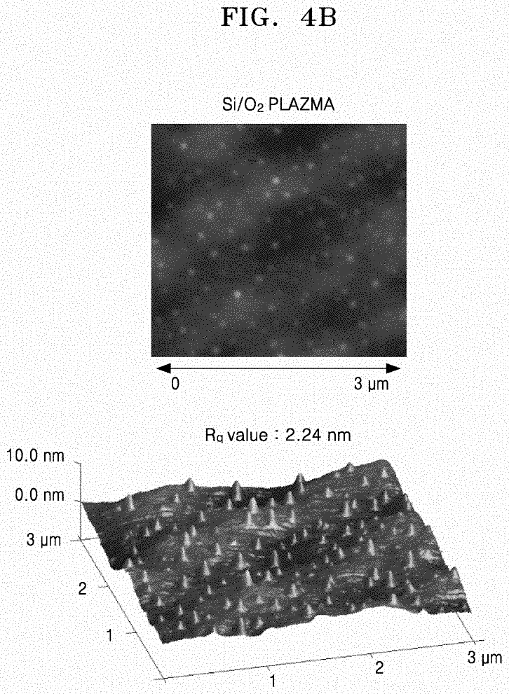

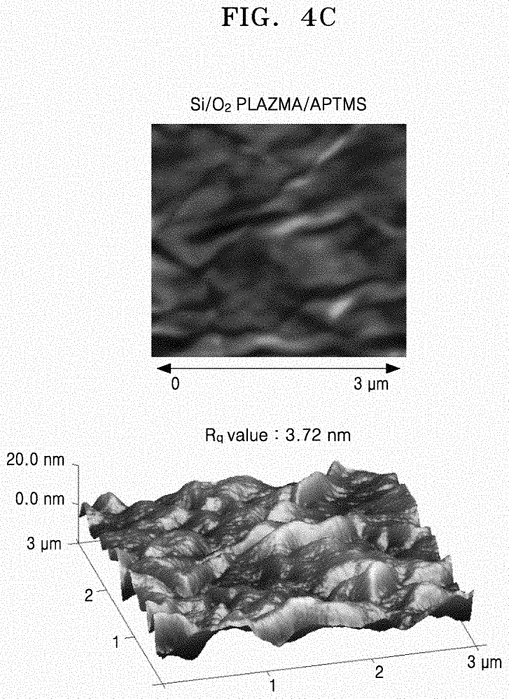

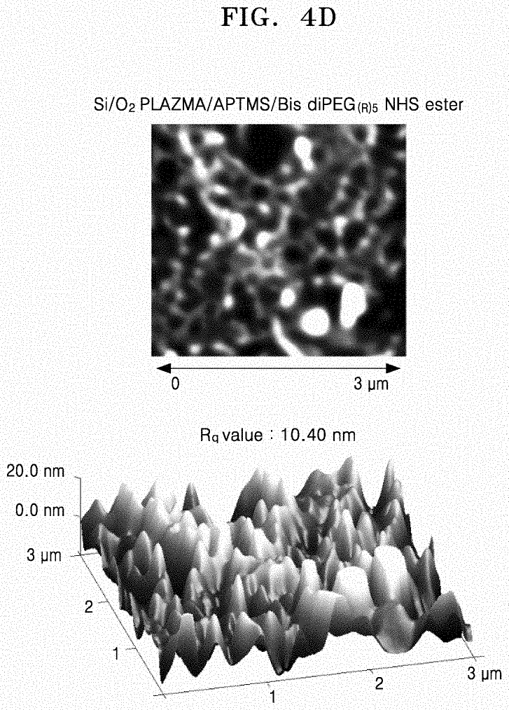

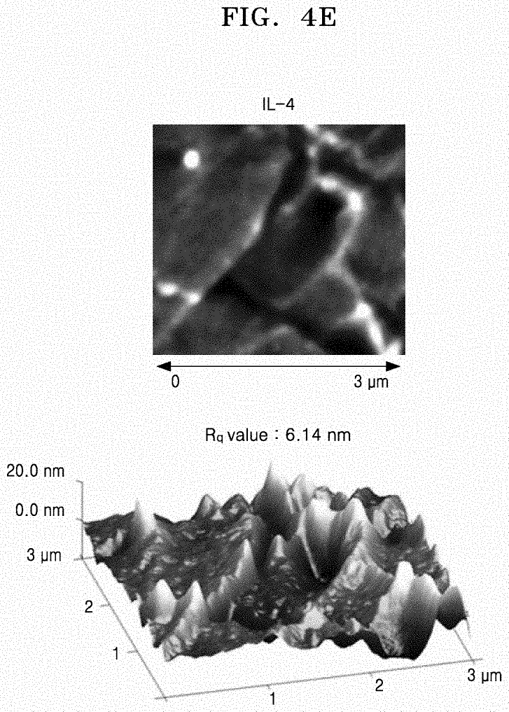

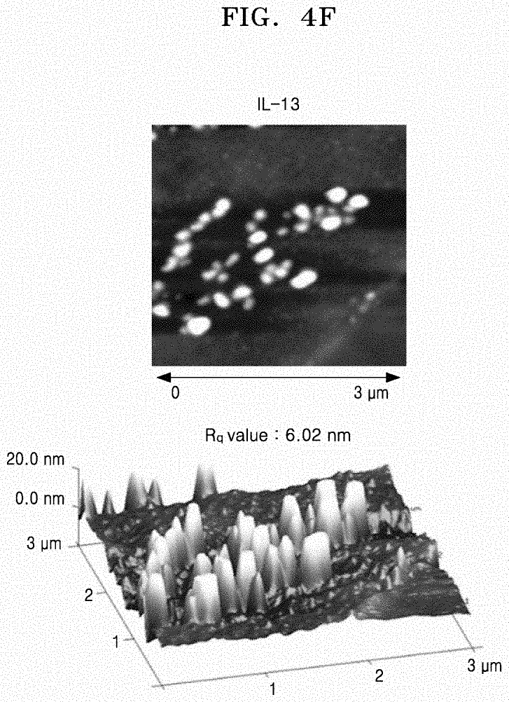

In this example, atomic force microscopy (AFM) analysis was performed in order to obtain an image of the surface layer of each sample used in Example 2. XE-100 AFM (Park Systems) was used for biofilm imaging, the resonant frequency was 200 kHz to 400 kHz, and the nominal force constant was set to be 42 N/m. Surface imaging was obtained in non-contact mode by using a silicone tip of a 125 μm-long nitride lever coated cantilever (PPP-NCHR 10M; Park Systems). The scan frequency was typically 1 Hz per line. Roughness was calculated with 3 μm×3 μm images. The results are shown in FIGS. 4A to 4F.

Regarding the results of FIGS. 4A to 4F, the surface mean square roughness (Rq) value of Si/O₂ Plasma/APTMS was slightly increased from 2.06 nm, which is the Rq value of the control group, to 3.72 nm, which is the Rq value of the APTMS treatment group, and after treatment with bis-dPEG®₅ NHS ester, the Rq value was increased to 10.4 nm. Additionally, as shown in FIGS. 4E and 4F, when IL-4 or IL-13 was added, the roughness values were decreased again to 6.14 nm and 6.02 nm, respectively. These results indicate that each of the additive materials successfully modified the silicon surface.

### EXPERIMENTAL EXAMPLE

### Experimental Example 1. Cell Viability Assay (MTT Assay)

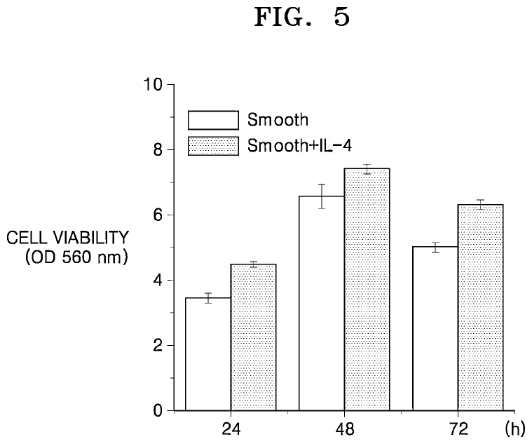

This experimental example was performed to measure the cytotoxicity of a medical implant according to an aspect. In order to measure cell viability as an indicator of cytotoxicity, RAW 264.7 cells were prepared by the method described in Reference Example 1. Cells were divided into two groups, which were then brought into contact with a smooth silicon surface which was not unmodified (smooth), or a silicon surface which was modified with IL-4 prepared according to Example 1 (smooth+IL-4). After detaching cells from each group at time points of 24, 48, and 72 hours, the cells were washed once with PBS. In DMEM medium, 0.5 mg/mL of MTT was added to each well, then the cells were incubated at a temperature of 37° C. for 4 hours, and then the MTT solution was removed. Finally, formazan crystals were dissolved in DMSO and the absorbance thereof at 560 nm was read in a microplate reader (EPOCH2, BioTek). The results are shown in FIG. 5.

According to the results shown in FIG. 5, the cell viability tended to increase from 24 hours to 48 hours, and at the time point of 72 hours, the cell viability was slightly decreased. This was the same in both groups. As shown in FIG. 4, the cells of the smooth+IL-4 group showed significantly better viability than the cells of the smooth group at all time points of 24, 48 and 72 hours. These results indicate that the cytotoxicity was significantly reduced by IL-4 introduced to the silicon surface.

### Experimental Example 2. Measurement of M1 and M2 Macrophage Differentiation

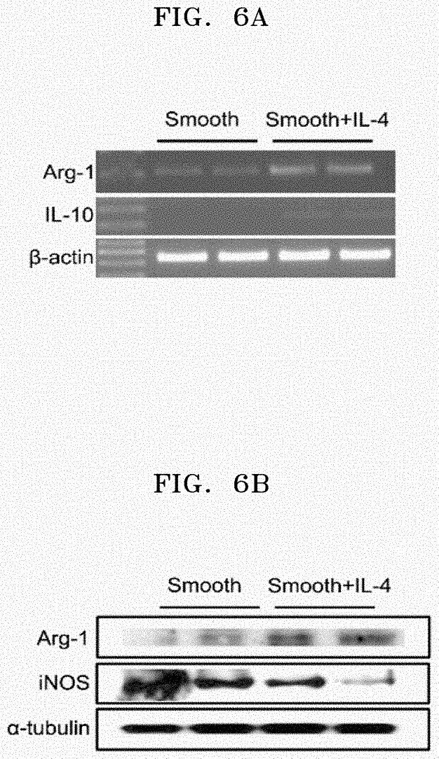

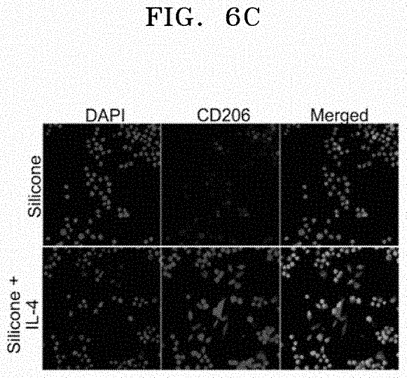

This experimental example was performed to identify whether IL-4 introduced to the silicon surface affects the healing of M2 or M1 wounds and tissue recovery of macrophages and affects the inflammatory immune response. After preparing RAW 264.7 cells by the method described in Reference Example 1, the cells were divided into two groups, which were respectively brought into contact with a non-modified smooth silicon surface, or a silicon surface modified with IL-4 prepared according to Example 1 (smooth+IL-4). Then, the immunofluorescence staining method of Reference Example 3 and Western blotting of Reference Example 2 were performed to confirm the expression levels of genes and proteins. The results are shown in FIGS. 6A to 6C.

As shown in FIGS. 6A and 6B, it was confirmed that Arg-1 and IL-10, which are markers of M2 macrophages, were more highly expressed in the smooth+IL-4 group. In addition, Western blot results showed that the M1 marker iNOS was expressed at a high level in the smooth group, and the M2 marker Arg-1 was significantly highly expressed in the smooth+IL-4 group. In addition, referring to FIG. 6C, the results of immunofluorescence staining showed that, in the smooth group, CD206 was hardly observed and, in the smooth+IL-4 group, CD206 was remarkably highly expressed. These results indicate that RAW 264.7 cells are more actively differentiated into M2 macrophages on the surface of IL-4 introduced silicon than in the smooth group.

### Experimental Example 3. Cytokine Production Measurement

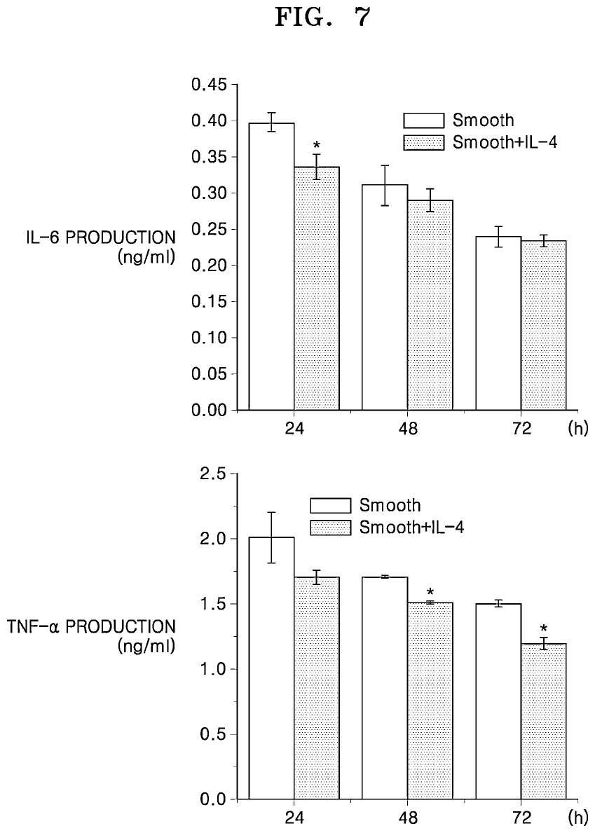

In this experimental example, the production of proinflammatory cytokines IL-6 and TNF-α was measured. Enzyme-linked immunosorbent assay (ELISA) was performed. Captured antibodies were diluted with PBS and coated on a 96-well plate at room temperature for 24 hours. Then, the plate was washed twice with PBS, and blocked with PBS with 10% FBS for 2 hours. After adding the sample extracted from the cell culture supernatant of each of the smooth group and the smooth+IL-4 group thereto, the reaction was performed at room temperature for 2 hours. After treatment with secondary antibodies, substrate reagents were reacted and reading was carried out at a 405 nm wavelength in an ELISA reader (EPOCH2, BioTek). The results are shown in FIG. 7.

As shown in FIG. 7, IL-6 tended to decrease over time, and from the first 24^{th} date, in the case of the smooth+IL-4 group, the detected concentration thereof was statistically significantly low. Regarding TNF-α, both groups showed the decreasing tendency over time, but at all time points, in the smooth+IL-4 group, the concentration thereof was significant low. These results indicate that IL-4-modified silicon can reduce the expression of pro-inflammatory cytokines, which could cause the generation of inflammation.

### Experimental Example 4. Measurement of Th2 Cell Cytokine Production

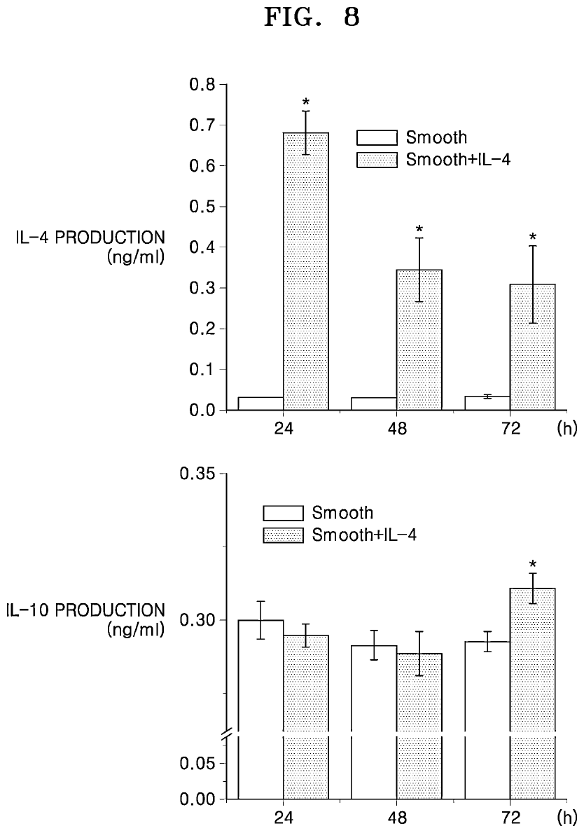

This experimental example was performed to measure the production of IL-4 and IL-10, which are anti-inflammatory cytokines. The measurement method was performed by the methods of RT-PCR and qRT-PCR described in Reference Example 4. The results are shown in the graph of FIG. 8.

As shown in FIG. 8, IL-4 was secreted significantly high in the Smooth+IL-4 group. In the case of IL-10, the two groups showed no difference after 24 and 48 hours, but after 72 hours, significantly high secretion occurred in the Smooth+IL-4 group. These results indicate that the Smooth+IL-4 group induced the expression of anti-inflammatory cytokines, and thus, the anti-inflammatory response and wound healing effects thereof were greater than those of the smooth group.

### Experimental Example 5. Measurement of STAT6 Pathway Activity

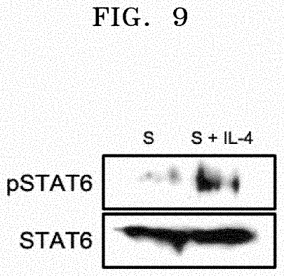

Activation of the STAT6 pathway is an important factor in differentiating macrophages to the M2 type. Accordingly, in this Experimental Example, Western blotting according to Reference Example 2. was performed on STAT6 and pSTAT6 to determine whether IL-4-modified silicon alone could activate the STAT6 pathway. The results are shown in FIG. 9.

As shown in FIG. 9, there was no significant difference in STAT6 between the Smooth+IL-4 group and the Smooth group. On the other hand, pSTAT6, which is the active form, was detected more in the Smooth+IL-4 group. These results indicate that in the smooth+IL-4 group, M2-type macrophages were generated more.

### Experimental Example 6. In Vivo Experiments

In this experimental example, an animal experiment was performed to measure the in vivo effect of the medical implant. For animal experiments, 10 Sprague-Dawley mice weighing 250 g to 300 g at 9 weeks of age were used. Five animals in each group were randomly distributed into each of two groups. Animals were exposed in a 12/12 h light/dark cycle in specific-pathogen-free (SPF) conditions with free access to food and water. Approval for this protocol was approved by the Bundang Seoul National University Hospital Animal Experiment and Use Committee (approval number: BA1801-240/011-01), and all procedures were in accordance with the guidelines of the NIH. There were an animal group in which an intact silicon was inserted as an implant and an animal group in which silicon modified with IL-4 was inserted as an implant. The former group was used as a control group.

The process of inserting the implant is specifically as follows. The subject mice were anesthetized by inhalation of isoflurane (Hana Pharm, Korea), the hair on the back was shaved, and the surgical site was disinfected with 70% alcohol and betadine. Then, a 2-3 cm incision was made in the back with a #15 scalpel blade, and the implant was inserted into the cortical pouch. The incision site was closed with surgical sutures (Nylon 4/0, Ethicon, USA). The surgical site was disinfected again with 70% alcohol and betadine and a light dressing was applied thereon.

Animals were monitored for 12 weeks after transplantation, confirming the development of cascade inflammation. Therefore, at predetermined time points of 1, 2, 4, 8 and 12 weeks, all animals in each group were tissue biopsied. For biopsies, selected animals were euthanized with carbon dioxide, and tissues and implants in the dorsal region with epidermis, dermis, posterior and anterior capsules were removed.

6.1. Evaluation of Capsular Thickness and Collagen Density In Vivo

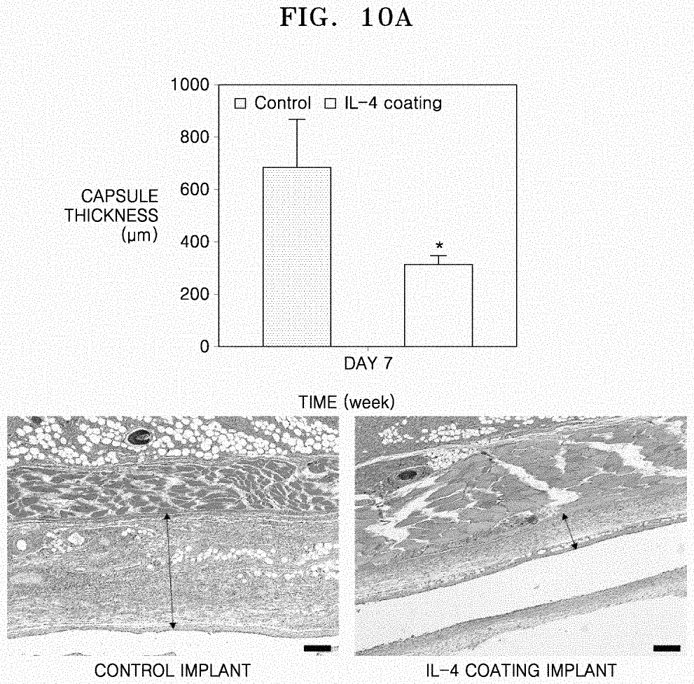

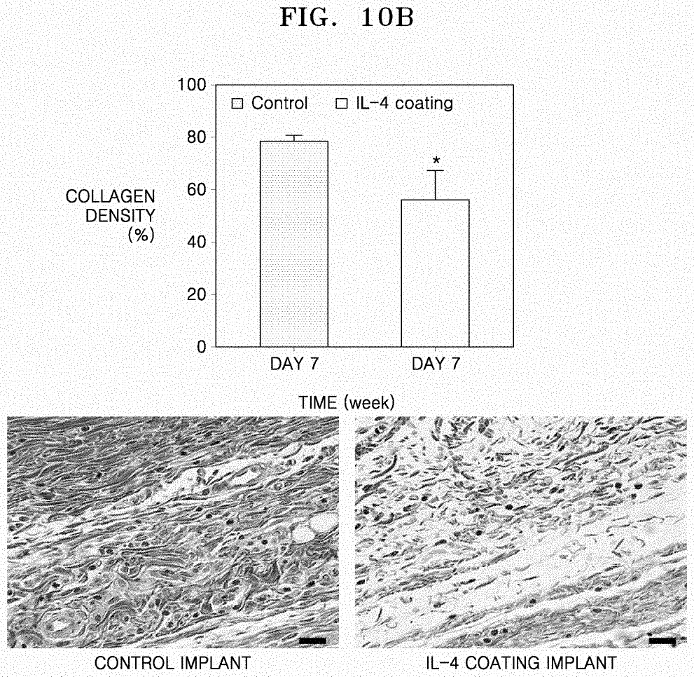

The thickness of the capsule tends to be increased over time due to the accumulation of collagen. Accordingly, the in vivo capsular thickness and collagen density were investigated. Capsular thickness was determined by analyzing tissue slides which were H&E-stained using a microscope (LSM 700, Carl Zeiss, Oberkochen, Germany) at 40× magnification. The capsular range was defined from the top of the silicone insertion area to the bottom of the dorsal subcutaneous muscle. To evaluate the overall capsular thickness from the tissue slides, three different parts of the capsule were randomly photographed, and the capsular thickness was measured with ZEN software. The results thereof are shown in FIG. 10A.

Collagen density was analyzed using image analysis of 5 randomly selected regions on slides stained with MT staining at 40× magnification. Collagen bundles were stained as being blue to analyze the density of collagen over the entire microscopic area with Image J software. The results thereof are shown in FIG. 10B.

Regarding the results of FIG. 10A, the capsular thickness of the control group was 688.5 μm±177.9 μm, whereas the capsular thickness of the mice implanted with IL-4 modified silicon was measured to be 317.8 μm±31.5 μm. It was confirmed that the group implanted with IL-4 modified silicon had a statistically significant inhibition of capsular formation on the 7^{th} day.

FIG. 10B shows that, regarding the density of capsule-constructing collagen, the collagen density of the group implanted with IL-4 modified silicon used as an implant was 56.5±10.8%, which was significantly reduced than the collagen density of the control group, which was 78.4±2.3%.

### Experimental Example 7. Evaluation of Biological Activity for IL-10 Surface-Modified Silicon Implants

The present experimental example was based on the IL-10 surface-modified silicon implant prepared in the same manner as in Example 1, and the levels of pro-inflammatory cytokines TNF-α, IL-6, and IL-13 produced in RAW 264.7 cells and the levels of IL-10 and IL-4 were measured. In addition, cytotoxicity evaluation with respect to IL-10 introduced into the silicon surface was performed, and the differentiation level of M2 or M1 macrophages was evaluated, and the effect on wound healing and tissue recovery of macrophages was confirmed. This experiment was performed in the same manner as in Experimental Examples 1 to 3, and the control group was a group (smooth) in which the subject was in contact with a smooth silicon surface, which was not modified.

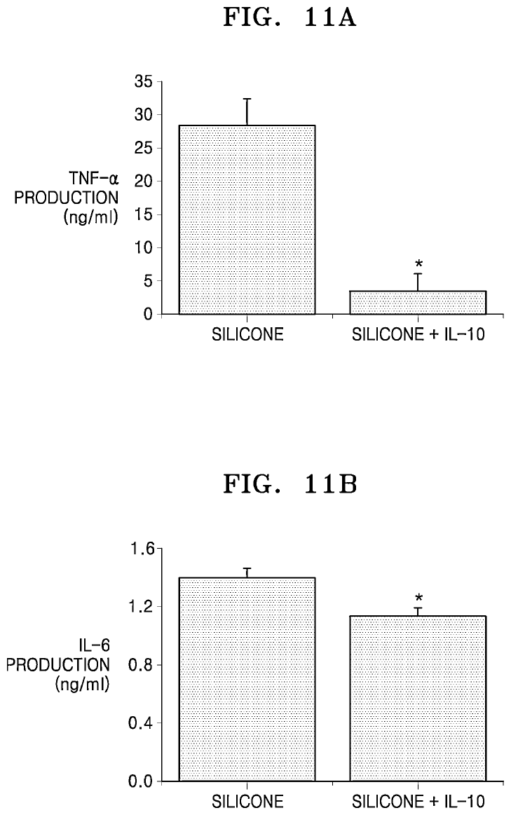

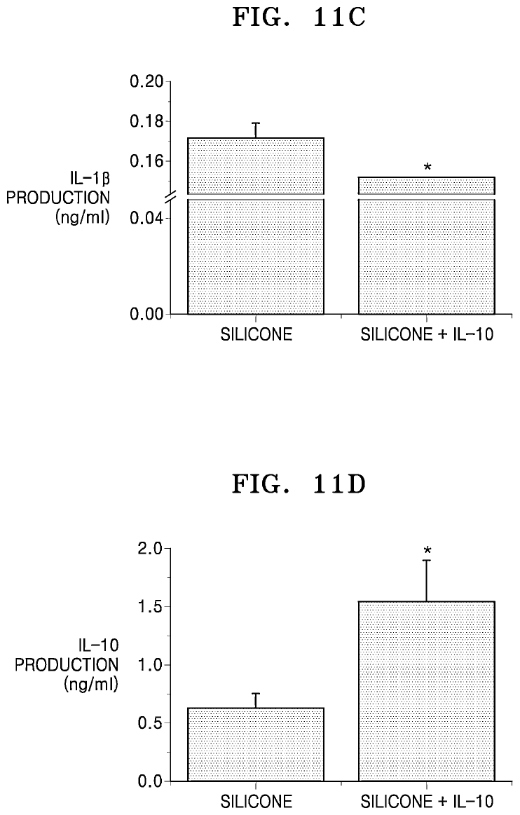

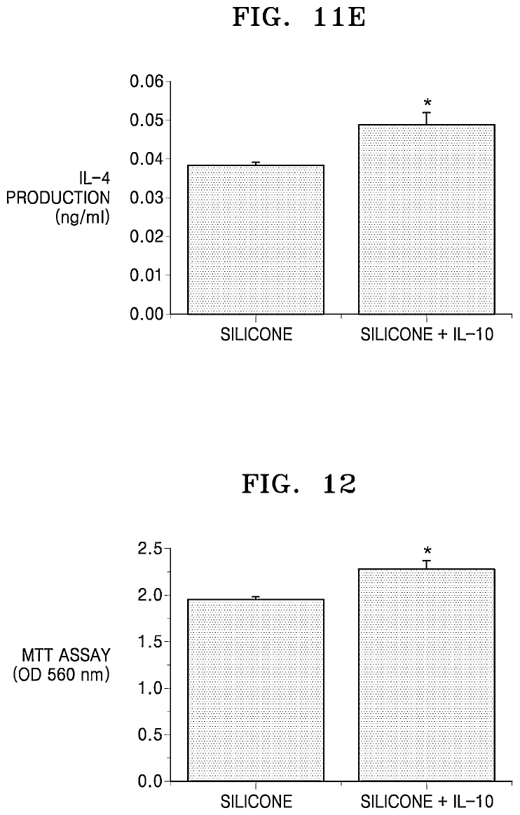

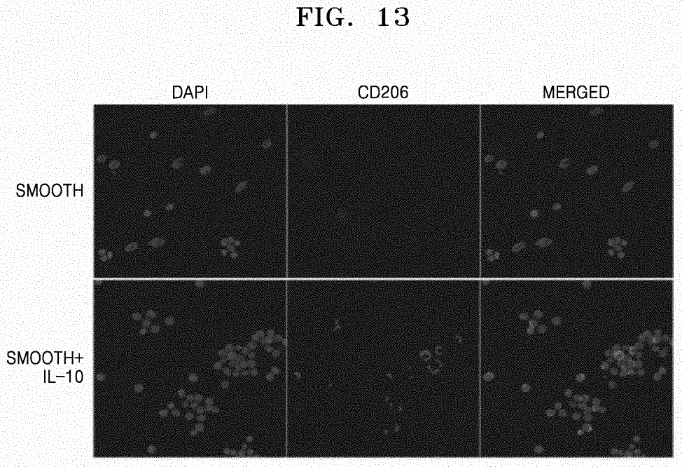

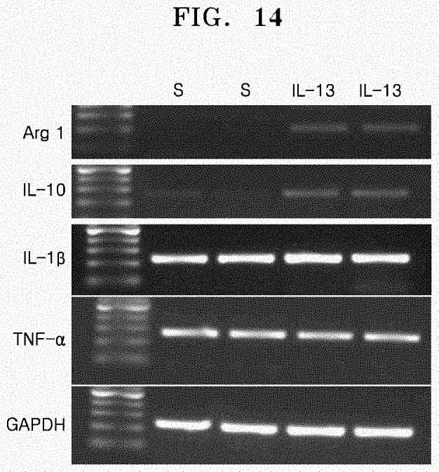

As a result, as shown in FIGS. 11A to 11E, the concentrations of TNF-α, IL-6, and IL-13 were significantly lower in the smooth+IL-10 group, whereas the concentrations of IL-10 and IL-4 were significantly higher in the smooth+IL-10 group. In addition, as shown in FIGS. 12 and 13, cytotoxicity did not occur in the IL-10-modified silicone, but rather showed higher viability compared to the smooth group, CD206, which is a marker indicating differentiation into M2 macrophages, and was not present in the smooth group, whereas was significantly highly expressed in the smooth+IL-10 group. These results indicate that IL-10 introduced into the silicon surface may reduce the side effects of in vivo transplantation by inhibiting the production of proinflammatory cytokines around the implanted site and promoting differentiation into M2 macrophages.

### Experimental Example 8. Biological Activity Evaluation of IL-13 Surface Modified Silicon Implants

In the present experimental example, regarding the IL-13 surface-modified silicon implant prepared in the same manner as in Example 1, the level of proinflammatory cytokines TNF-α, and IL-1β produced in RAW 264.7 cells and the level of IL-10 were measured, and the level of Arg-1, a marker of M2 macrophages, was measured. In addition, cytotoxicity evaluation with respect to IL-13 introduced to the silicon surface was performed. This experiment was performed in the same manner as in Experimental Examples 1 to 3, and the control group was a group (smooth) in which the subject was in contact with a smooth silicon surface, which was not modified.

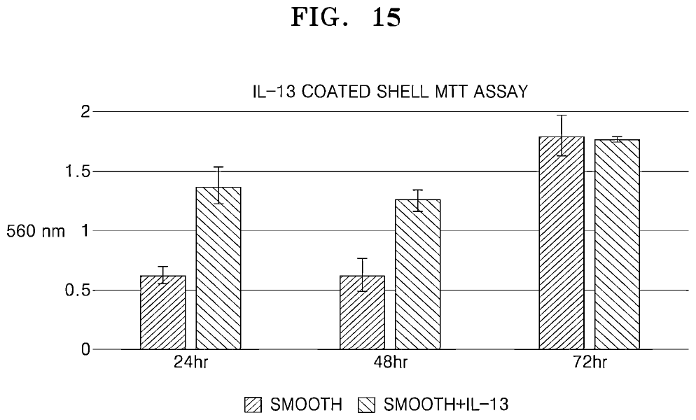

As a result, as shown in FIG. 14, the level of TNF-α in the smooth+IL-13 group (IL-13) was decreased, while the levels of Arg-1 and IL-10 were increased. In addition, as shown in FIG. 15, the smooth+IL-13 group showed better viability than the cells of the smooth group at the time points of 24 and 48 hours. These experimental results show that IL-13 can also contribute to reducing the side effects caused by implantation in vivo, although the degree of the reducing was smaller than that of IL-4.

The description of the present disclosure described above is for illustration, and those of ordinary skill in the art to which the present disclosure pertains will understand that it can be easily modified into other specific forms without changing the technical spirit or essential features of the present disclosure. Therefore, it should be understood that the embodiments described above are illustrative in all respects and not restrictive.

1. A medical implant comprising:
an implant base having a surface made of a silicon material;
a linker having one end attached onto the surface of the implant base; and
a functional polypeptide bound to another end of the linker.

2. The medical implant of claim 1, wherein the surface of the implant base includes a shell of a silicon material.

3. The medical implant of claim 1, wherein the functional polypeptide is at least one selected from a cytokine and a chemokine.

4. The medical implant of claim 3, wherein the cytokine is at least one selected from interleukin-4 (IL-4), interleukin-10 (IL-10), and interleukin-13 (IL-13).

5. The medical implant of claim 1, wherein the linker is represented by Formula 1: 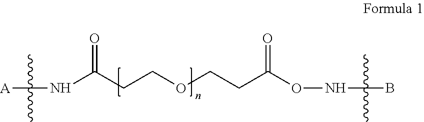
wherein, in Formula 1,
A is an implant with a silicon surface,
B is the end of the functional polypeptide, and
n is an integer from 5 to 15.

6. The medical implant of claim 1, wherein the medical implant induces secretion of an anti-inflammatory cytokine.

7. The medical implant of claim 1, wherein the medical implant is a breast implant.

8. The medical implant of claim 7, wherein the breast implant suppresses the formation of breast capsular contracture.

9. The medical implant of claim 6, wherein the breast implant has a roughness value (Rq) of about 4 nm to about 10 nm.
